(19) United States
(12) Patent Application Publication
(10) Pub. No.: US 2016/0067103 A1
(43) Pub. Date: Mar. 10, 2016

(54) THERMOPLASTIC SHEET FOR MOLDING A MASK ON A PERSON'S BODY

(71) Applicants: TOMTEC NV, Kapellen (BE); ANTHONY MEDICAL & MARINE TECHNOLOGIES BVBA, Kapellen (BE)

(72) Inventor: Jean-Michel Anthony, Kapellen (BE)

(21) Appl. No.: 14/784,397

(22) PCT Filed: Apr. 11, 2014
(86) PCT No.: PCT/EP2014/057373

§ 371 Date: Oct. 14, 2015

(30) Foreign Application Priority Data
Apr. 16, 2013 (EP) 13164006.2

(51) Int. Cl. A61F 13/00; A61N 1/04

(52) U.S. Cl. CPC A61F 13/00063; A61F 13/00987; A61F 13/00017; A61F 13/00038; A61F 13/00029; A61F 13/00991; A61N 1/0468; A61F 2013/00157

(57) ABSTRACT

The present invention provides a thermoplastic sheet material for skin cosmetic treatment and/or skin medical treatment, comprising a thermoplastic composition layer having an upper surface and a lower surface; and a support layer bonded on at least one surface of the thermoplastic composition layer, wherein said support layer comprises silicone.

### TECHNICAL FIELD

The present invention concerns a thermoplastic sheet material. More in particular, it concerns a thermoplastic sheet material suitable for directly molding a mask on at least part of a person's body.

### BACKGROUND

Thermoplastic materials are well-known in their medical use as shaped articles for the purpose of immobilizing an anatomical region of the body. These materials are also used in the medical field to manage dermal scarring from traumatic injuries such as burns, particularly of the face. The contours of the mold may be sculpted and the thermoplastic may be reshaped from time to time as to guide the patient's soft tissue into forming desired contours.

WO 2013/001083 provides a thermoplastic sheet material, comprising a thermoplastic composition layer and a bonded support layer comprising an elastic fabric. The support layer is continuously bonded on the thermoplastic sheet material by heat-bonding or using an adequate adhesive. The thermoplastic sheet material provides is suitable to be used in a method for molding anatomical shaped medical articles, in particular face masks and braces.

Bonding a support layer to a thermoplastic composition layer by heat bonding technique presents several disadvantages. Indeed, in order to obtain high-performance assembly, the thermoplastic composition layer must meet specific requirements. For instance, excessive cleaning and surface preparation of the thermoplastic composition layer should be performed. Furthermore, a narrow temperature range is to be used thermal bonding. If the temperature is too low, there is inadequate bond strength. If the temperature is too high, the thermoplastic composition layer will melt excessively and the properties of the thermoplastic composition layer will be altered. Consequently, the obtained thermoplastic sheet will have low moldable properties and the obtained mask will not properly fit to the patient's body part to be treated.

In addition, heat bonding usually requires applying a pressurized quenching to consolidate the bonding layer and to cause the quick hardening of the heated thermoplastic composition. This slows down the production process and increases production costs.

Another problem occurs when wearing a thermoplastic material, especially on sensitive skin such as the skin of the face. The direct contact of the thermoplastic material on the skin causes complaints or even skin irritation. Face masks for supporting the administration of medicines for example have to be worn for a long time and therefore need optimal comfort.

The present invention aims to resolve at least some of the problems mentioned above.

The invention thereto aims to provide a thermoplastic sheet material suitable to be directly molded on a patient's body. The thermoplastic sheet material is as described in claim 1.

### SUMMARY OF THE INVENTION

In a first aspect, the present invention provides a thermoplastic sheet material for skin cosmetic treatment and/or skin medical treatment, comprising a thermoplastic composition layer having an upper surface and a lower surface; and a support layer bonded on at least one surface of the thermoplastic composition layer, wherein said support layer comprises silicone. Preferably, said support layer comprises a silicone layer which is directly bound to at least one surface of the thermoplastic composition layer. Said silicone layer is in direct contact with the surface of the thermoplastic composition layer and hence with the thermoplastic composition layer.

In a preferred embodiment, the support layer further comprises a compound selected from the group comprising silver ions, silver bromide, silver carbonate, silver chloride, silver nitrate, silver sulfadiazine, a biocide, an anti-bacterial or any combination thereof.

In a preferred embodiment, the support layer is bonded by spraying said support layer on at least one surface of the thermoplastic composition layer.

In a preferred embodiment, the support layer further comprises a coating on the surface which is not bonded to the thermoplastic composition layer. In a preferred embodiment, the coating comprises a synthetic polymer.

In a preferred embodiment, the sheet material is moldable at a low temperature which is comprised between 40° C. and 90° C., preferably between 50° C. and 70° C. and remains in plastic condition until cooled below 30° C. In a preferred embodiment, the sheet material is rigid at hardened condition.

In another aspect, the present invention provides a method for skin cosmetic treatment and/or skin medical treatment comprising the step of directly molding a mask on at least a portion of a person's body using a thermoplastic sheet material as described above.

The present invention provides for the use of a thermoplastic sheet material as described above for molding directly on a person and/or for molding a mask, whereby the support layer is the skin-facing layer. This feature is advantageous as direct molding on the person provides a personalized mask. In use, said personalized mask and in particular the thermoplastic composition layer of the mask, applies a pressure on the person's skin and/or scar. More in particular, the rigidity of the mask applies a pressure on the person's skin and/or scar. The latter optimizes the treatment thereby leading to a shortening of the treatment time. The action mode of the molded mask is then comparable to the action mode of compression bandages.

The present invention provides for the use of a thermoplastic sheet material as described above for skin cosmetic treatment and/or skin medical treatment of burns or dermatologic scars.

In another aspect, the present invention provides a method for the production of a thermoplastic sheet material as described above, comprising the step of spraying a support layer on at least one surface of the thermoplastic composition layer. In a preferred embodiment, the support layer comprises silicone. Preferably, the support layer comprises a silicone layer which is directly bound to at least one surface of the thermoplastic composition layer.

The present invention further provides a method for the production of a thermoplastic sheet material as described above, comprising the step of bonding the support layer to at least one surface of the thermoplastic composition layer using UV curing. In a preferred embodiment, the support layer comprises silicone. Preferably, the support layer comprises a silicone layer which is directly bound to at least one surface of the thermoplastic composition layer.

The present invention presents several advantages. The thermoplastic sheet can be covered by a very thin layer of silicone thanks to the use of the spray technique. Thin layers of silicone are usually difficult and bonding a thin silicone layer to a thermoplastic layer frequently leads to a non-uniform bonding. In addition, the silicone layer creates a microclimate that enhances the penetration of a compound into the person's skin thereby shortening the treatment period and leading to optimized results.

### DESCRIPTION OF FIGURES

FIG. 1 shows a top view of a preferred embodiment of a thermoplastic sheet material according to the invention.

FIG. 2 illustrates a cross section A-A of a preferred embodiment of a thermoplastic sheet material bonded with a coated support layer according to the invention.

FIG. 3 depicts a cross section A-A of a preferred embodiment of a thermoplastic sheet material bonded with an uncoated support layer according to the invention.

FIG. 4 depicts a preferred embodiment of the method of softening a thermoplastic sheet material according to the invention.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight” (weight percent), here and throughout the description unless otherwise defined, refers to the relative weight of the respective component based on the overall weight of the formulation.

As used herein the terms “thermoplastic sheet material” and “thermoplastic sheet” are synonyms.

As used herein the terms “brace” and “splint” or “cast” are used as synonyms.

In a first aspect, the present invention provides a thermoplastic sheet material 1 for skin cosmetic treatment and/or skin medical treatment, comprising a thermoplastic composition layer 3 having an upper surface 4 and a lower surface 5; and a support layer 6 bonded on at least one surface of the thermoplastic composition layer 3, wherein said support layer 6 comprises silicone.

In a preferred embodiment, the present invention provides a facial mask for skin treatment wherein said mask is produced by directly molding the thermoplastic sheet material 1 on the patient's face.

### Thermoplastic Composition Layer

By the term “thermoplastic composition layer” 1 as used herein, is understood a layer comprising a thermoplastic composition 3 having two surfaces: an upper surface 4 and a lower surface 5. The thermoplastic composition 3 may be chosen from the group of polycaprolactone and polyurethane or any combination thereof.

The polyurethane may be present in an amount of 0%, 10%, 20%, 30%, 40% or 50% (% by weight), preferably 20% to 40% or a value in the range between any two of the aforementioned values, most preferably 30%. The polycaprolactone may be present in an amount of 60%, 70%, 80%, or 90% (% by weight) or a value in the range between any two of the aforementioned values, preferably 60% to 80%, most preferably 70%. Typically, there will be more polycaprolactone than polyurethane which polycaprolactone lowers the temperature at which the sheet deforms. The ratio of polycaprolactone:polyurethane (weight:weight) may be 5:1, 4:1, 3:2, 3:1, 2.3:1, 2:1 preferably 2.3:1.

The molecular weight of the polyurethane may be equal to or less than 10000, 20000, 30000, 40000, 50000, 60000, 70000, 80000, 90000, 100000, 120000, 140000, 150000 or a value in the range between any two of the aforementioned values, preferably between 10000 and 100000. Polyester polyurethane is the preferred polyurethane.

The molecular weight of the polycaprolactone may be 10000, 20000, 30000, 40000, 50000, 60000, 70000, 80000, 100000, 200000, 300000, 400000, 500000 or a value in the range between any two of the aforementioned values, preferably between 10000 and 60000, more preferably between 37000 and 500000.

Caprolactone polyester polyurethane is particularly suitable, which polyurethane may be obtained by reacting isocyanate and polycaprolactone-based polyester. Such a caprolactone polyester polyurethane is commercially available as a granulate. The melting point of said polycaprolactone polyester polyurethane lies between 190° C. and 210° C. By adding the polycaprolactone, also preferably in granulate form, a thermoplastic composition is obtained that is distortable and moldable at a temperature of about 69° C. and remains distortable by cooling down about 30° C.

At this temperature, the thermoplastic composition layer 3 may be stretched at least up to twenty times the original length thereof. In the hardened condition, the thermoplastic material is rigid and has a memory effect that, after heating, said material returns to its original shape, so to its shape before molding. It is non-elastic in the hardened condition. The thermoplastic composition layer of the present invention is used to be directly molded on a human body part thereby obtaining anatomical shaped medical articles (corresponding to the thermoplastic composition layer at the hardened condition).

The thermoplastic composition layer 3 may have a thickness of 0.8 mm, 1.0 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm or 6.0 mm or a value in the range between any two of the aforementioned values.

In one embodiment, the thermoplastic composition layer 3 comprises between 1% to 40% (weight %) of microspheres of non-metallic, heat-accumulating material which is especially suited for heating in a micro-wave oven. Preferred are glass microspheres with a diameter between 20 μm and 800 μm. A coloring agent may be added to the composition.

The thermoplastic composition layer 3 is preferably perforated to increase breathability. The perforations 2 may have a diameter of 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2.0 mm or a value in the range between any two of the aforementioned values.

A non-limiting example of a thermoplastic composition that can be used in the current invention is Turbocast® (T Tape Company BV).

### Support Layer

By the term “support layer” 6 is meant a layer that comprises a composition different from the thermoplastic composition layer 3 and supports the thermoplastic composition layer 3. In a preferred embodiment of the invention, the thermoplastic composition layer 3 comprises a support layer 6 only on one of its surfaces 4 or 5. In another embodiment of the invention, the thermoplastic composition layer 3 comprises support layers 6 on both of its surfaces 4 and 5. In a preferred embodiment, the support layer comprises silicone. Preferably, said support layer comprises a silicone layer which is directly bound to at least one surface of the thermoplastic composition layer. By directly bound, it is meant that the silicone layer is in direct contact with the surface of the thermoplastic composition layer. In a further preferred embodiment, the support layer consists of silicone.

The advantage of using silicone as support layer is that said silicone will act as a friction restraining material and will provide comfort to the wearer when the silicone side is the skin facing side of the sheet. Silicone reduces the friction between the sheet and the skin. Another advantage of silicone is its heat resistance capacity; this will buffer the heat of the thermoplastic sheet material 1 when it is directly molded on the skin. Another advantage of silicone is that it is vapor-permeable. Furthermore, a major advantage of using silicone is that it creates a micro-climate where it is applied. So when the mask is used for the face, a microclimate is created between the face skin and the silicone of the mask. Such micro-climate enhances penetration into the skin of the patient of compounds and/or active molecules present in the silicone. Furthermore, said micro-climate will allow for an accelerated healing of burns and or dermatologic scars and it will lessen permanent scars on the skin. This enhances the treatment efficiency and shortens the treatment time. The microclimate created by the silicone enhances treatment by 10 to 20 times, preferably by 15 times compared to a treatment wherein such microclimate is not created. Another advantage of the microclimate created by the silicone is to provide reproducible treatment thereby offering constant conditions for a treatment comprising repetitive use of the mask. By treatment, we refer to cosmetic treatments, medical treatments or any other treatments known to the person skilled in the art and requiring the immobilization of at least a part of the patient's body.

The silicone further enhances the memory effect of the thermoplastic sheet. As previously mentioned, in the hardened condition or after molding on a patient body part, the thermoplastic material returns to its original shape, so to its shape before molding, after heating it. Silicone applied in relatively thin layer to the thermoplastic sheet provides more elasticity to the sheet thereby enhancing its memory effect. Preferably, the thickness of the silicone is of from 10 to 60 microns, preferably from 20 to 50 microns, more preferably about 40 microns.

The silicone is preferably non-adhesive. The silicone can also be adhesive on at least one of its sides. The adhesive side can then be used to bond the silicone to the thermoplastic composition layer. The silicone can also be partially adhesive thereby comprising adhesive and non-adhesive areas. The silicone is preferably washable which reduces contamination when the treatment requires repetitive use of the mask. Preferably, the silicone is translucent.

In a preferred embodiment, the silicone is provided with high tear strength. Preferably, the gravity of said silicone, determined according to ASTM D792, is of from 0.5 to 2 gr/cm³, more preferably from 0.8 to 1.5 gr/cm³, most preferably about 1 gr/cm³.

In a preferred embodiment, the shore A of the silicone, determined according to ASTM D2240, is of from 40 to 100, preferably from 45 to 85, more preferably from 50 to 70, most preferably about 60.

In a preferred embodiment, the tensile strength of the silicone, determined according to ASTM D412, is of from 5 to 12 Mpa, preferably from 6 to 10 Mpa, more preferably from 7 to 9 Mpa, most preferably about 8 Mpa.

In a preferred embodiment, the tear strength of the silicone, determined according to ASTM D624—method A, is of from 10 to 25 KN/m, preferably from 12 to 20 KN/m, more preferably from 14 to 17 KN/m, most preferably about 15 KN/m.

In a preferred embodiment, the elongation of the silicone, determined according to ASTM D412, is of from 400 to 900%, preferably from 500 to 800%, more preferably from 600 to 700%.

In a preferred embodiment, the tack free time, determined according to ASTM C679/NT-TM005, is of from 2 to 20 min, preferably from 5 to 18 min, more preferably from 8 to 16 min, most preferably from 10 to 12 min.

In a preferred embodiment, the temperature of fragility of the silicone is of from 100 to 400° C., preferably from 150 to 350° C., more preferably from 180 to 250° C., most preferably from 200 to 220° C.

In another preferred embodiment, the support layer comprises a layer of non-adhesive material such as polyurethane or any other material known to the person skilled in the art. Said layer is obtained by spraying a polyurethane water based suspension on the thermoplastic composition layer.

In a preferred embodiment, the support layer 6 further comprises a compound selected from the group comprising silver ions, silver bromide, silver carbonate, silver chloride, silver nitrate, silver sulfadiazine, a biocide, an anti-bacterial or any combination thereof. In a preferred embodiment, the thermoplastic sheet comprises about 3% of said compound. In a preferred embodiment, the support layer consists of silicone and any compound listed in the above mentioned group.

In a preferred embodiment of the invention, the thermoplastic composition layer and/or the support layer comprises a compound selected from the group comprising silver ions, silver bromide, silver carbonate, silver chloride, silver nitrate, silver sulfadiazine, a biocide, an anti-bacterial or any combination thereof. In a preferred embodiment, the thermoplastic sheet comprises about 3% of said compound.

In a preferred embodiment, the support layer may contain other metals such as gold (in form of glitters or filaments), platinum, zirconium, copper or any combination thereof.

The use of the aforementioned compounds is advantageous compared to the use of nanotechnologies and/or nanoparticles for cosmetic treatments and/or for medical treatment. Indeed, secondary and/or side effects of nanotechnologies and/or nanoparticles it is unknown while it is well proves that the use of the aforementioned compounds and in particular, the silver compounds, has no side and/or secondary effects.

Silver in its elemental form in particular has a very low solubility product, meaning that, compared with other active antimicrobial ingredients; a very low concentration of active ingredient is released in the presence of wound exudate. Yet this concentration is sufficient to provide an antimicrobial effect within a wound dressing. Without wishing to be tied to the theory, it is believed that silver bound within a wound dressing, develops its effect in elemental form at the level of the support layer and in the form of silver-complex ions or silver ions detaches out of the support layer by the wound exudate. Thus, the silver can act within the wound dressing only except for a very low concentration, preventing any contamination with excessive amounts of silver on the wound.

In addition, the support layer enhances the hygiene of the mask. For instance, when the mask is used for cosmetic purposes or for any other purpose, it is frequently the case that the user skin covered by the mask is sweating. As mentioned above, the silver of the support layer provides an antimicrobial effect, thereby enhancing the hygiene of said mask.

In a preferred embodiment, the support layer releases less than 25 μg of silver per 1 cm² of support layer surface per 24 hours at 25° C. in 100 ml of water or physiological saline, more particularly less than 15 μg of silver per 1 cm² of support layer surface per 24 hours at 25° C. in 100 ml of water or physiological saline and more particularly less than less than 5 μg of silver per 1 cm² of support layer surface per 24 hours at 25° C. in 100 ml of water or physiological saline.

By the term “breathable” it is meant that air can pass through the fabric and/or the material.

As used herein, the term “breathable” also refers to a material which is permeable to water vapor. Breathable fabrics of the present invention have a water vapor transmission rate WVTRs comprised between 1 and 2000 g/m²/24 hours, preferably between 2 and 1800 g/m²/24 hours, more preferably between 3 and 1600 g/m²/24 hours, most preferably between 3 and 1400 g/m²/24 hours.

In a preferred embodiment of the invention, the support layer is suitable for freely following the deformations of the thermoplastic composition layer 3, during and after molding of the thermoplastic sheet material. The support layer will give support to the thermoplastic sheet material 1 when it is in moldable phase but during hardening and molding of the thermoplastic composition, the support layer freely follows the expansions and contractions of the thermoplastic composition. When the thermoplastic composition is rigid, the support layer is fully adapted to the form of the thermoplastic sheet material 1.

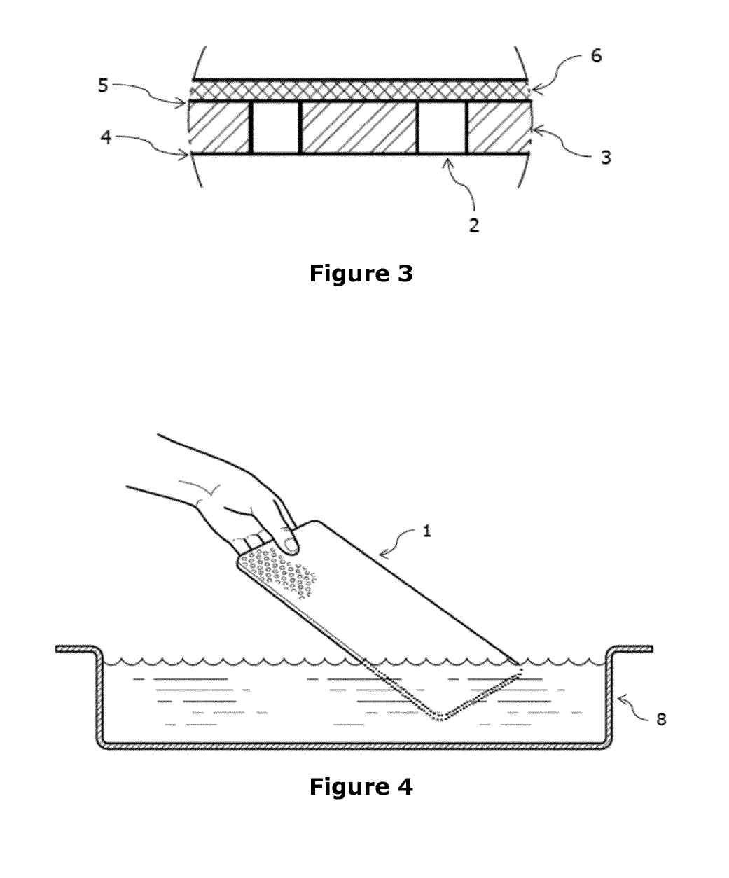

In one embodiment of the invention, the support layer 6 is uncoated. Such embodiment is depicted in FIG. 3.

In a second aspect, the invention relates to a thermoplastic sheet material 1, comprising a thermoplastic composition layer 3 bonded with a support layer 6 which is coated.

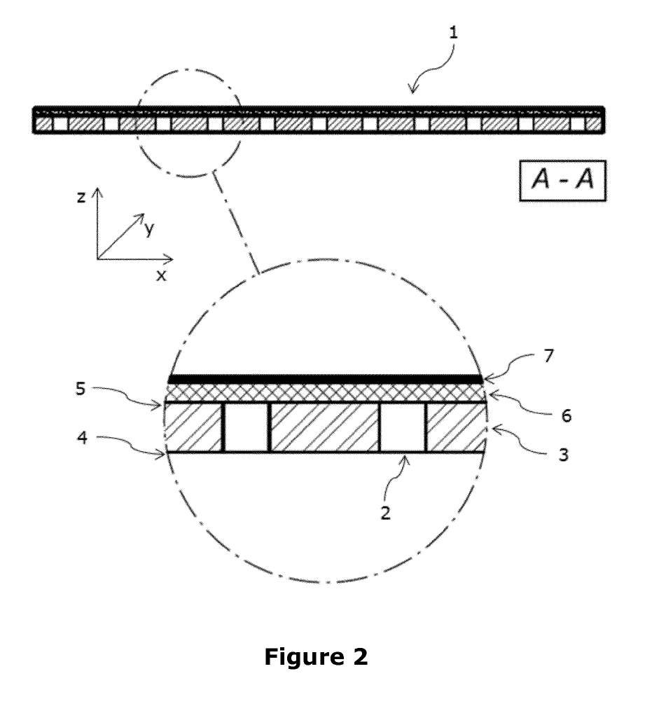

In an embodiment of the invention, the support layer 6 is coated with a polymer as in FIG. 2. By preference, the polymer is chosen from the group comprising but not limited to elastomers such as ethylene-vinyl acetate, ethylene propylene rubber, polyester, breathable polyurethane; and synthetic silicone. The support layer 6 may be coated on one side or on both sides. The advantage of using polymer coating 7 is that it will freely follow the expansion and contraction of the thermoplastic sheet material.

In a preferred embodiment of the invention, the support layer 6 optionally comprises a backing layer which is removed before application to the subject. The backing layer reversibly adheres to the support layer intended for contact with the skin of the subject. The backing layer may be made of any suitable material such as, for example, polyethylene, polythene or other polymeric substance. According to an aspect of the invention, the backing layer is smooth, or is regularly patterned with pits or grooves. In another preferred embodiment of the invention, the coating 7 optionally comprises a backing layer. Said backing layer of the coating is similar to the one described for support layer.

In another embodiment of the invention, the support layer can be impregnated. By preference, the support layer is impregnated with a polymer. More by preference, the polymer is chosen from the group comprising but not limited to elastomers such as ethylene-vinyl acetate, ethylene propylene rubber, polyester.

In a preferred embodiment, the present invention provides a breathable thermoplastic sheet comprising a thermoplastic composition layer 3, a support layer 6 bonded on one surface of the thermoplastic composition layer 3. The thermoplastic composition layer 3 is preferably provided with perforations. Said support layer 6 is preferably polyurethane open cell foam which is coated and/or impregnated with a breathable silicone elastomer. Thus, the thermoplastic sheet exhibit good breathability and improved wear comfort which is advantageous for the user. It is to be understood that the thermoplastic composition layer can also be devoid of perforations.

The thickness of the support layer 6 is equal to or less than 0.025 mm, 0.05 mm, 0.06 mm, 0.08 mm, 0.1 mm, 0.5 mm, 0.8 mm, 1 mm, 1.2 mm, 1.5 mm, 2.0 mm, 2.5 mm, 3.0 mm, 3.5 mm, 4.0 mm, 4.5 mm, 5.0 mm or a value in the range between two of the aforementioned values.

### Bonding of Support Layer on Thermoplastic Composition Layer

In another aspect, the present invention provides a method for the production of a thermoplastic sheet material as described above, comprising the step of spraying a support layer 6 on at least one surface of the thermoplastic composition layer. In a preferred embodiment, said support layer comprises silicone. Preferably, said support layer comprises a silicone layer which is directly bound to at least one surface of the thermoplastic composition layer. The support layer is in direct contact with the surface of the thermoplastic composition layer.

In a preferred embodiment, the support layer 6 is sprayed on the thermoplastic composition layer 3, thereby coating at least one surface of said thermoplastic composition layer. Said spraying can be performed at room temperature. Spraying can be performed using any method known to the person skilled in the art. Preferably, the support layer is sprayed on said thermoplastic composition layer using a spray gun. Using spray method, a thin support layer can be easily created on the thermoplastic composition layer. This is not offered by other bonding methods wherein handling of thin support layers is delicate and leads to irregularities in the final produced material.

In a preferred embodiment, silicone is sprayed on the thermoplastic composition layer and is allowed to cool and dry thereby forming a first layer of silicone covering at least one surface of the thermoplastic composition layer. A second layer of silicone can be created on the silicone layer which is already obtained. Said second layer is also created by spraying silicone using a spray gun. Said second layer with said first layer determine the thickness of the support layer applied on the thermoplastic composition layer. This provides a control over the total thickness of the thermoplastic sheet material.

In a preferred embodiment, the support layer 6 is deposited on the thermoplastic composition layer 3. After deposit, the support layer is scraped using a scraping means thereby coating at least one surface of said thermoplastic composition layer with an equally distributed support layer. The support layer might be deposited by spraying it on the thermoplastic composition layer. Here also, a thin support layer can be easily created on the thermoplastic composition layer.

In a preferred embodiment, the thermoplastic composition layer 3 is treated with an adhesion primer prior to the application and/or the creation of the support layer 6. Said primers enhance the adhesion of the support layer to the thermoplastic composition layer. Said primers can be of chemical and/or mechanical nature or any other type known to the person skilled in the art.

In a preferred embodiment of the invention, 70%, preferably 80%, more preferably 90%, most preferably 100% of the surface area of at least one side of the thermoplastic composition layer 3 is coated by the support layer 6. Both sides of the thermoplastic composition layer 3 might be coated by the support layer 6.

In the method of the present invention, the support layer 6 comprising silicone might be applied to the thermoplastic composition layer using different techniques. Said support layer might be applied by screen printing or by roller coating.

The present invention further provides a method for the production of a thermoplastic sheet material as described above, comprising the step of bonding the support layer to at least one surface of the thermoplastic composition layer using ultraviolet (UV) curing. In a preferred embodiment, the support layer comprises silicone. Preferably, the support layer comprises a silicone layer which is directly bound to at least one surface of the thermoplastic composition layer. The support layer is in direct contact with the surface of the thermoplastic composition layer.

A silicone layer is placed on at least one surface of the thermoplastic composition layer. The laminated structure is placed under UV lights to be cured. Preferably, the curing time is of from 10 to 120 seconds, more preferably from 20 to 100 seconds, most preferably from 30 seconds to 80 seconds, most preferably from 40 to 60 seconds. Amongst the advantages of UV curing are the facility of the application and treatment and the short operation time which increases production speed. In addition, using UV curing method, there is no need for heating the laminate thereby preserving the thermoplastic composition layer from any deformation. Said thermoplastic composition layer being moldable at low temperatures as mentioned above.

### Thermoplastic Sheet Material

By the term “thermoplastic sheet material” 1 as used herein, is understood a sheet material that is deformable under the application of heat and after cooling down retains the deformed shape and becomes rigid. The overall thickness of the sheet material 1 may be 1.5 mm, 1.6 mm, 1.7 mm, 1.8 mm, 1.9 mm, 2.0 mm, 2.2 mm, 2.4 mm, 2.5 mm, 2.6 mm, 2.7 mm, 2.8 mm, 2.9 mm, 3.0 mm, 3.2 mm, 3.4 mm, 3.5 mm, 3.6 mm, 3.7 mm, 3.8 mm, 3.9 mm, or 4.0 mm or a value in the range between any two of the aforementioned values. In one embodiment of the invention, the thickness of the thermoplastic sheet material 1 is higher than 4.0 mm, for example, 4.1 mm, 4.5 mm, 5.0 mm, 5.5 mm, 6.0 mm, 7.0 mm, 8.0 mm or a value in the range between any two of the aforementioned values.

The thermoplastic sheet material 1 according to the invention is by preference moldable at temperatures of between 40° C. and 90° C., more by preference between 50° C. and 70° C. This temperature is depending on the polycaprolactone content of the sheet material. The sheet material will remain in a plastic condition until it is cooled below 30° C. Typically the sheet is brought into the moldable state by heating in a water bath 8 set around 5° C. above the melting temperature, most preferable at 65° C. Such method is depicted in FIG. 4. Alternatively, the sheet material 1 can be heated using a convection or fan oven, a microwave oven or a hair dryer or by any other heat producing apparatus or method.

Perforations 2 with a diameter of at least 0.5 mm and preferably between 1.0 to 2.0 mm, or a value in the range between any two of the aforementioned values may be provided in the thermoplastic sheet material, so as not to hamper the skin breathing after applying the sheet.

When handling an unsupported thermoplastic sheet while it is in a moldable state, initial deformations occur in the thermoplastic composition 3, such as impression of fingers or gloves which make contact with the sheet. An unsupported thermoplastic sheet also deforms under its own weight when it is reheated prior to molding. A preferred embodiment of the support layer according to the invention will counteract to these deformations due to the presence of a support layer consisting of silicone. As a result, a thermoplastic sheet according to the invention will keep its original form until it is molded during its plastic phase.

The characteristics of the support layer will allow for wanted deformation of the thermoplastic material upon application of external forces, such as during molding.

The thermoplastic sheet 1 remains by preference in the moldable state for 1-10 minutes, more by preference between 2-5 minutes. The support layer 6 is capable of freely following the expansion and the contraction of the thermoplastic composition 3 while the thermoplastic composition hardens during and after molding. Such sheet 1 can directly be molded on a person and is suitable for use as a mask, brace, splint or cast material for immobilizing a part of a person. The support layer is by preference the skin-facing side while the skin non-facing side comprises by the thermoplastic sheet material.

In an embodiment of the invention, the thermoplastic sheet material 1 comprises a Turbocast® thermoplastic composition on which silicone layer is disposed.

The sheet, according to the present invention, can directly be molded on a person and is suitable for the use as a face mask whereby the support layer will be the skin-facing layer. The resulting face mask is suitable to be used for skin treatment. Said face mask provides a comfortable wearing and the support layer 6 prevents adhesion to the skin and/or hair by the thermoplastic composition 3. The sheet 1 exhibits excellent deformability properties, conforming to the shape of the face without the need to apply excessive pressure. The silicone layer provides a comfortable wearing against the skin.

In a preferred embodiment, the face mask comprises further at least one aperture for the nose and/or at least one aperture for the mouth and/or at least one aperture for the eyes. Said mask may comprise at least one fixture for attachment to a strap. Prior molding, the mask is provided as a pre-cut thermoplastic sheet material.

The sheet, according to the present invention, can directly be molded on any part of a person's body whereby the support layer will be the skin-facing layer. The obtained mask can hence be a face mask; a mask covering only a part of the face; a mask covering the face, the neck and at least part of the shoulders; a mask covering any other part of the person's body.

In a preferred embodiment, a preformed cast is used during molding of the sheet of the present invention on a person's body part. Preferably the molded thermoplastic material and the cast cover the full body part. Said preformed cast is designed to cover the anatomical contours of a first area of said body part. The anatomical contours of a second area of said body part is then covered by the molded sheet. Said second area is not covered by the cast. The second part of the body part is the part to be treated and will be covered by the molded thermoplastic sheet material. The use of a preformed cast during the molding of the sheet immobilises the body part of interest thereby avoiding movement of said body part during molding. This increases the molding precision thereby leading to a mask that is perfectly conforming and covering the body part to be treated. The cast is used for molding the thermoplastic sheet to on the body art to be treated. After molding the thermoplastic sheet, the obtained mask can be used and attached to the patient body part without the use of said preformed cast.

The cast is used to assist the practitioner molding the thermoplastic sheet on the body part to be treated. Preferably, the thermoplastic sheet to be molded is suitable to be attached to the cast on at least 2, 4, 6 or 8 different attachment points. The attachment might be performed using hook and loop fastener system, press-stud system or snap-fastener system. For molding the thermoplastic sheet on the body part of interest, the practitioner can attach the sheet to the cast on one or two different points. Afterwards, the sheet can be molded on the body part and attached to the each of the remaining attachment points of the cast. If needed, a slight pressure can be applied on the sheet during molding thereby obtaining a mask that perfectly conforms the contours of the body part to be treated. The presence of attachment points on the cast allows one practitioner to mold the thermoplastic sheet. There is no longer a need for more than one person to hold the sheet during molding. The use of the cast thereby considerably lowers the number of personnel required to mold the thermoplastic sheet on a body part to be treated.

In a preferred embodiment, the preformed cast is suitable for embracing and supporting a body part of a human. Said cast consists of a pre-formed sheet of material comprising cork and a thermoplastic binder. Said cast is preferably flexible. A particular characteristic of the cast is that although it is flexible, it provides sufficient rigidity to the body part to be treated.

The cast can be made of a thermoplastic material. Preferably, the cast is made of a material comprising a combination of cork and a thermoplastic binder; such as preferably ethylene vinyl acetate (EVA). Cork is a moisture-adsorbent material, while the applied thermoplastic binder is a moisture repellent material. Combining two materials having different and contradicting properties leads to a cast having optimal characteristics of hardness and flexibility. The cast is made of flexible material such that it is suitably deformable in order to conform to contours of body part to be treated.

The cast can be perforated. Preferably, the perforations cover about 75% to 95% of the cast area. The material of the cast, in particular the cork, is breathable thereby making said cast comfortable to wear. The cast allows perspiration/transpiration. Problems of skin irritation or skin maceration can be avoided.

In a preferred embodiment, the cast is made of a flexible material comprising cellulose, preferably cork, and a binder, preferably a thermoplastic binder.

The use of cork in the cast has the advantage that cork has initial strength. The cork thus contributes to the stability and strength of the cast. Cork is a natural flexible and tough component which makes the material in combination with a thermoplastic binder, such as e.g. EVA—very suitable for functional bracing or splinting. Furthermore, cork is a natural and environmental friendly component.

The binder is preferably a thermoplastic binder. Suitable thermoplastic polymers include polyurethanes (especially polyurethanes based on semi-crystalline polyester polyols), polyethylene, ethylene vinyl acetate (EVA), cis and trans polyisoprene, polyesters such as polycaprolactone and the like. The currently preferred binder is a rubber, such as a synthetic rubber, preferably an acetate polymer and most preferably EVA (ethyl vinyl acetate). EVA is durable plastic foam material made of ethylene vinyl acetate resin. EVA-containing material is sufficiently deformable, but still offers adequate rigidity to the cast. In practice the cast containing EVA material will easily fit the contour of the body part to which is it fixed. EVA feels soft on the skin, does not induce allergic reactions on the skin and is X-ray translucent. The material can be advantageously made at low production costs and has a natural appearance.

In a particularly preferred embodiment, the cast is made of a preformed sheet of material comprising cork and EVA. Preferably, the used material contains at least 20 weight % of cork, and at least 40 weight % of EVA. In an example the present material comprises 1 kg of cork for 4; 4.5; 5; 5.5; 6; 6.5; 7; 7.5; or 8 kg of EVA.

The combination of cork and EVA gives the material unique characteristics which cannot be gained with a combination with other material such as for instance cotton or cellulose with EVA. The combination of cork with EVA makes it possible to control both strength and flexibility. Furthermore the combination makes the cast water repellent which make the brace reusable.

In another preferred embodiment, the cast material may further contain additives and/or colorants. In a preferred embodiment, the additives may comprise metal oxides. In an example, the present cast consists of cork material that has been pulverized and mixed with acetate polymers and ethyl vinyl in the presence of the metal oxide additives such as silicium oxide (preferably 15-20% w/v), aluminium oxide (preferably 5-10% w/v), zinc oxide (preferably 1-2% w/v), iron oxide (preferably 0.5-1% w/v), sodium, potassium and calcium oxide (preferably 0.1-0.5% w/v), and titan dioxide (preferably 0.01-1% w/v). The material may be provided as foamed and cross-linked microcellular solid with or without colorants. This material may be extruded and/or injected, e.g. in a pre-formed mold or the like.

In a preferred embodiment, the cast softening temperature is preferably between 75 to 130° C., and more preferably about 80 to 110° C.

In a preferred embodiment, the mask obtained after molding the thermoplastic sheet is provided with at least one fixture for a strap. It is to be understood that said fixtures are provided for attaching the mask to the body part to be treated. Attachment of the mask to a body part to be treated applies a light pressure on said body part. The position and the number of said fixtures depend on the body part to be treated. The fixtures for the strap may be a single slot, or two or more slots, however, it is not necessarily limited thereto. The fixtures may comprise one part of a hook and loop fastener, or one part of a press-stud or snap-fastener, which attaches to a reciprocating element on the strap. The fixtures are adapted to affix a strap that passes around the back of the head, and secures the mask to the face of the subject. The strap is preferably made from an elasticated substance, such as a strip of elasticated material, and may include a length adjuster, to adjust the length of the strap and thus the pressure exerted by the mask on the face.

In a preferred embodiment, the straps are attached to the mask in a configuration wherein at least part of the strap is surrounding at least partially the mask sheet, for instance when both parts of a hook and loop fastener, of a press-stud or of a snap-fastener are provided on the strap itself. Said strap is introduced in the fixture and both parts of the hook and loop fastener, the press-stud or the snap-fastener are fixed to each other. This is advantageous as the pressure applied on the wearer's face is smaller compared to the pressure applied by a mask in which the straps are fixed on top and/or on the side of the mask which is not in contact with the wearer's face. In the latter case, the straps will push the mask on the wearer's face which will be uncomfortable for said wearer. In the configuration described in the frame of the present invention, the straps do not push the mask on the wearer's face but will only secure the mask to wearer's face.

In a preferred embodiment, the thermoplastic sheet material and/or the mask obtained after molding the thermoplastic sheet is connectable to at least one electrode which provides electro-stimulations to the body part. Said electrode is connectable to a generator and can be attached to the thermoplastic sheet material and/or the mask by any suitable means which are known to the person skilled in the art. Preferably, the sheet and/or the mask is connectable to 2, 3, 4 or 5 electrodes. The generator produces electrical impulses and the electrodes provide electrostimulation treatment by stimulating the skin and subcutaneous muscles.

Wherein the thermoplastic sheet material comprises perforations, the electrode can be attached such as to be in contact with the sheet surface which is covering the treated body part. The electrode can be placed in between the thermoplastic composition layer and the support layer. Said electrode can also be attached to the skin-facing surface of the support layer. Preferably, the size of the electrode is larger than the size of the perforations. The electrode is connected to a generator via at least one electric wire which is entered through at least one perforation of the sheet or of the mask. Due to the size difference, the electrode is prevented from passing through the perforation and is thereby fixed to the body part by the mask or the sheet. Preferably, the size of the electrode is at least 1.1 to 4 times higher than the size of the perforation, more preferably between 1.5 and 2 times higher than the size of the perforation. Preferably, the sheet and/or the mask is connectable to a number of electrodes comprised between 1 and 30, preferably between 5 and 25 electrodes. The generator produces electrical impulses and the electrodes provide electrostimulation treatment by stimulating the skin and subcutaneous muscles. In a preferred embodiment, the number of electrodes and/or the position of each electrode is suitable to be changed within the mask according to the zone of the face to be treated. This is possible thanks to the perforations of the mask which allow for connecting electrodes to said mask as described above. This provides the user with a freedom to select the face zone to be treated. The user can treat the same zone or different zones during consecutive use of the mask.

In a preferred embodiment, the mask of the present invention is reusable from 2 to 30 times, preferably from 5 to 25 times, more preferably from 10 to 20 times.

In a preferred embodiment, the thermoplastic sheet material or the mask obtained after molding the thermoplastic sheet comprises a pulsed electromagnetic field (PEMF) transducer. Said transducer comprises at least one transducer winding for generating a PEMF stimulation signal in response to bi-directional current through the winding. The transducer winding might be attached to any side of the thermoplastic sheet which is coated or not coated with a support layer. Said winding can also be incorporated, during the production process, into the thermoplastic sheet material. The pulsed electromagnetic field (PEMF) transducer provides pulsed magnetic fields to the body part to be treated which is covered by the mask thereby treating injuries and/or stimulating cellular repair and/or regeneration.

In another aspect, the present invention provides a method for skin cosmetic treatment and/or skin medical treatment comprising the step of directly molding a mask on at least a portion of a person's body using a thermoplastic sheet material 1 as described above. The mask is molded by heating the thermoplastic sheet material in a water bath 8 as described above. The heated sheet is placed on the person's body part, for instance the face, such as the silicone layer is in contact with the skin. The sheet will deform according to the person's face. The sheet is allowed to cool down and to cure. This method provides each person with a personalized mask that can be stored for future use. Preferably, a preformed cast is used during molding of the sheet of the present invention on a person's body part. The preformed cast is as described above. In a preferred embodiment, the method comprises the step of providing electro-stimulations to the treated body part.

In a preferred embodiment, during heating of the thermoplastic sheet, a signal is provided to the user or the practitioner when the temperature of the sheet is appropriate for molding. Said signal is preferably a visual signal such as a change of the color of the sheet.

In a preferred embodiment, the silicone comprises a compound intended to be delivered to the person's skin. The silicone creates a microclimate that enhances the penetration of said compound into the person's skin. Said penetration might take place during the curing of the thermoplastic sheet and hence during the production of the personalized mask. In a different scenario, the personalized mask is produced, the silicone is then impregnated with said compound and the mask is places on the person's face. Still in a different scenario, the compound is applied directly on the person's face and the mask is then placed on the face for enhancing penetration of said compound into the person's skin.

A thermoplastic sheet material 1 according to the invention is suitable to be used in cosmetic treatments and/or in the treatment of burns and/or dermatological scars. Additionally, the support layer may contain anti-bacterial products and/or any compound as described above.

In another embodiment of the invention, a thermoplastic sheet material 1 according to the invention is suitable to be used for cosmetic skincare. Additionally, the support layer 6 may be treated with cosmetic products, suitable to be used in cosmetic therapy.

In another embodiment of the invention, a thermoplastic sheet material 1 according to the invention is suitable to be used for treatment of dermatological scars after breast operations. A thermoplastic sheet material 1 can be included in a bra whereby the silicone layer is facing the skin of the patient. The inclusion of a thermoplastic sheet material according to the invention in a bra will fasten the healing process of the skin after such operation and will lessen the amount and grade of the scars.

In an embodiment of the invention, the thermoplastic material may be pre-formed prior to molding. In another embodiment of the invention, the thermoplastic sheet material 1 is available in sheets with standard dimensions.

In an embodiment of the invention, the thermoplastic material may be sealed prior to use. This sealing is preferably sterile, vacuum and water-repellent. Such embodiment is suitable to use for treatment of burns and dermatological scars. Such embodiment of a thermoplastic sheet material 1 is by preference heated while hermetically sealed and opened in a sterile environment just before application to the skin that needs to be treated. The sealing is by preference watertight.

The invention is further described by the following non-limiting examples which further illustrate the invention, and are not intended to, nor should they be interpreted to, limit the scope of the invention.

It is supposed that the present invention is not restricted to any form of realization described previously and that some modifications can be added to the presented example of fabrication without reappraisal of the appended claims.

### EXAMPLES

### Example 1

### Thermoplastic Sheet Material

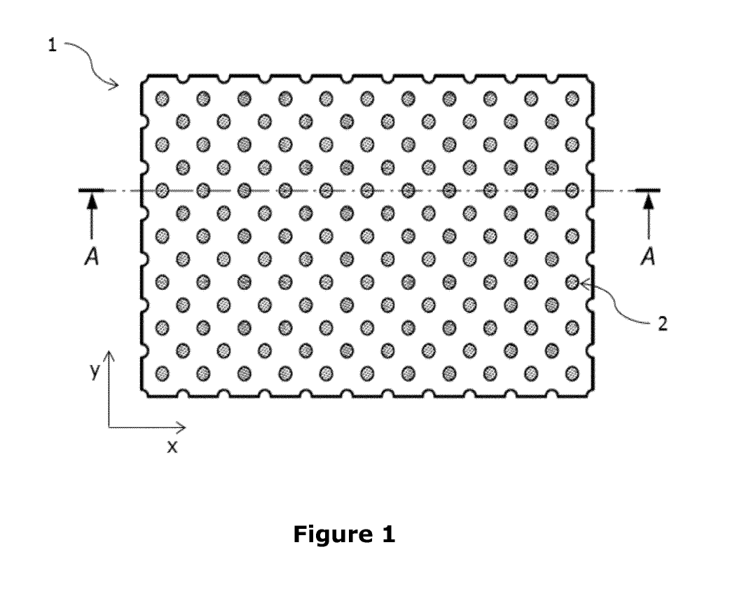

A preferred embodiment of the invention is depicted in FIG. 1 and has a thermoplastic composition layer 3 with following properties:
material: 30% polyester polyurethane, 70% polycaprolactone
melting point: 200° C.
thickness: 2 mm
perforations: 0.7 mm diameter

This composition is sprayed on its lower surface 5 with silicone having the following properties:
material: synthetic silicone
coating weight: 12 g/m²
application: sprayed on one side of the thermoplastic composition layer 3.

### Example 2

### Face Skin Treatment Using Mask

The mask of the present invention was used for treatment of the face skin with an active ingredient which reduces wrinkles. Two mask of the invention were used wherein the first mask comprises a support layer consisting of silicone and the support layer of the second mask consists of silicone and silver ion. In parallel and in the same conditions, the same active ingredient was applied on a face skin using a mask having the same composition as the mask of the present invention but devoid of silicone.

The treatment included 4 repetitions wherein in each repetition the active ingredient was applied for 15 minutes to the skin before being washed. The amount of active ingredient applied on the skin was 0.5 g/cm² or 1 g/cm² of skin.

The results were expressed percentage of treated wrinkles including wrinkle size reduction, wrinkle depth reduction and wrinkle number reduction. The obtained results are shown in table 1.

| | | | Use of mask: | | | |
|---|---|---|---|---|---|---|
| Repeti- | Use of mask: | | silicon + | | Mask without | |
| tion | silicon | | ion silver | | silicone | |
| number | 0.5 g/cm² | 1 g/cm² | 0.5 g/cm² | 1 g/cm² | 0.5 g/cm² | 1 g/cm² |
| 1 | 0.6% | 1.3% | 0.6% | 1.3% | 0.2% | 0.7% |
| 2 | 1.2% | 2.5% | 1.2% | 2.5% | 0.8% | 1.1% |
| 3 | 1.8% | 3.1% | 1.8% | 3.1% | 1.2% | 1.9% |
| 4 | 2.2% | 5.1% | 2.2% | 5.1% | 1.6% | 2.6% |

Table 1 clearly shows that the use of the mask comprising silicone as provided by the present invention leads to more efficient treatment. Indeed, the percentage of treated wrinkles after using the mask with silicone was always higher compared to the treatment wherein a mask devoid of silicone was used.

In addition, the obtained results show that better results are achieved using the mask provided with silicone even when a small quantity of active ingredient is used. The results obtained using a mask provided with silicone and 0.5 g/cm² of active ingredient were approximately similar to the results obtained using a mask devoid of silicone and 1 g/cm³ of active ingredient.

What is claimed is:

1. A thermoplastic sheet material for skin cosmetic treatment and/or skin medical treatment, comprising a thermoplastic composition layer comprising an upper surface and a lower surface; and a support layer bonded on at least one surface of the thermoplastic composition layer, wherein said support layer comprises a silicone layer which is in direct contact with the surface of the thermoplastic composition layer.

2. A thermoplastic sheet material according to claim 1, wherein said support layer further comprises a compound selected from the group consisting of ions, silver bromide, silver carbonate, silver chloride, silver nitrate, silver sulfadiazine, a biocide, an anti-bacterial and any combination thereof.

3. A thermoplastic sheet material according to claim 1, wherein said thermoplastic composition layer further comprises a compound selected from the group consisting of silver ions, silver bromide, silver carbonate, silver chloride, silver nitrate, silver sulfadiazine, a biocide, an anti-bacterial and any combination thereof.

4. A thermoplastic sheet material according to claim 1, wherein the support layer is bonded to the thermoplastic composition layer by UV curing.

5. A thermoplastic sheet material according to claim 1, wherein the support layer is bonded by spraying said support layer on at least one surface of the thermoplastic composition layer.

6. A thermoplastic sheet material according to claim 1, wherein said support layer further comprises a coating on the surface which is not bonded to the thermoplastic composition layer.

7. A thermoplastic sheet material according to claim 1, wherein said sheet material is connectable to at least one electrode thereby providing electro-stimulation to the skin.

8. A thermoplastic sheet material according to claim 1, wherein the sheet material is moldable at a temperature between 40° C. and 90° C., preferably between 50° C. and 70° C. and remains in plastic condition until cooled below 30° C.

9. A thermoplastic sheet material according to claim 1, wherein the sheet material is rigid at hardened condition.

10. A method for skin cosmetic treatment and/or skin medical treatment comprising directly molding a mask on at least a portion of a person's body using a thermoplastic sheet material as described in claim 1.

11. (canceled)

12. The method according to claim 10, wherein the support layer is the skin-facing layer.

13. A method of treating skin or burns or dermatologic scars comprising directly molding a mask on at least a portion the skin of a subject in need thereof, wherein the mask comprises the thermoplastic sheet material of claim 1.

14. A method for the production of a thermoplastic sheet material as described in claim 1, comprising spraying a support layer on at least one surface of the thermoplastic composition layer.

15. A method for the production of a thermoplastic sheet material as described in claim 1, comprising UV curing said support layer on at least one surface of the thermoplastic composition layer.

16. The method of claim 10, wherein the treatment is for a burn or scar.

17. The method of claim 10, further comprising electro-stimulating the skin by connecting the sheet material to at least one electrode.

18. The method of claim 13, wherein the support layer is the skin-facing layer.
